(19) United States
(12) Patent Application Publication
(10) Pub. No.: US 2019/0091615 A1
(43) Pub. Date: Mar. 28, 2019

(54) Method And Apparatus For Preparation Of A Urea Solution

(71) Applicant: Tenneco Automotive Operating Company Inc., Lake Forest, IL (US)

(72) Inventors: Michael C. BRADFORD, Chelsea, MI (US); Alan C. BROCKMAN, Ann Arbor, MI (US); Ryan A. FLOYD, Mason, MI (US)

(21) Appl. No.: 15/712,253

(22) Filed: Sep. 22, 2017

(51) Int. Cl. B01D 39/16; F01N 3/20; F01N 3/28; B01D 53/94; B01D 15/10; B01J 23/04; B01J 27/232; B01J 35/04; B01J 16/00; C07C 273/16

(52) U.S. Cl. CPC B01D 39/16; F01N 3/208; F01N 3/2896; B01D 53/9418; B01D 15/10; B01D 2239/0407; B01J 27/232; B01J 35/04; B01J 16/00; C07C 273/16; F01N 2610/02; B01J 23/04

(57) ABSTRACT

An exhaust after-treatment system and a method for treating an engine exhaust that each utilize a filter for removing biuret from an aqueous urea exhaust treatment fluid. The filter includes a filter element that includes an adsorbent material configured to adsorb the biuret from the aqueous urea exhaust treatment fluid, and includes a biuret conversion catalyst impregnated in the adsorbent material that is configured to convert the biuret into a material useful for exhaust after-treatment or into a material that is innocuous to an exhaust after-treatment system.

### FIELD

The present disclosure relates to a method and apparatus for preparing a urea solution.

### BACKGROUND

This section provides background information related to the present disclosure which is not necessarily prior art.

Stringent emissions legislation in Europe and North America is driving the implementation of new exhaust after-treatment systems. Exhaust after-treatment technologies are currently being developed that will treat nitrogen oxides (NOx) under these conditions. One of these technologies includes a catalyst that facilitates the reactions of ammonia (NH₃) with the exhaust NOx to produce nitrogen (N₂) and water (H₂O). This technology is referred to as Selective Catalytic Reduction (SCR).

Ammonia is difficult to handle in its pure form in the automotive environment, therefore it is customary with these systems to use a liquid aqueous urea reagent solution, typically at a 32.5% concentration of urea (CO(NH₂)₂), commonly known as diesel exhaust fluid (“DEF”) and by its commercial name of AdBlue®. The urea is delivered to the hot exhaust stream and is transformed into ammonia prior to entry in the catalyst.

Commercially available aqueous urea reagent solutions such as AdBlue®, however, can also include impurities. One such impurity is biuret. When biuret is exposed to elevated temperatures of the exhaust, solid deposits can form on exposed surfaces of the exhaust after-treatment system that can interfere with the proper operation of the exhaust after-treatment system. These deposits can be found throughout the system, including on the exhaust pipe walls and sometimes on the SCR component in the exhaust passage. If left untreated, these deposits can negatively affect performance of the exhaust after-treatment system.

### SUMMARY

This section provides a general summary of the disclosure, and is not a comprehensive disclosure of its full scope or all of its features.

The present disclosure provides an exhaust after-treatment system for treating an exhaust produced by an engine. The exhaust after-treatment system includes an exhaust passage in communication with the engine that is configured to carry the exhaust, a DEF delivery system including an injector that is configured to dose an aqueous urea exhaust treatment fluid into the exhaust passage, and a tank in communication with the injector that is configured to provide the aqueous urea exhaust treatment fluid to the injector. A filter is located within the DEF delivery system, and the filter is configured to remove impurities from the aqueous urea exhaust treatment fluid, wherein one of the impurities of the aqueous urea exhaust treatment fluid is biuret, and the filter includes an adsorbent material configured to adsorb the biuret from the aqueous urea exhaust treatment fluid, and the filter includes a biuret conversion catalyst impregnated in the adsorbent material that is configured to convert the biuret into a material useful for exhaust after-treatment or into a material that is innocuous to the exhaust after-treatment system.

The present disclosure also provides a method for treating an exhaust produced by an engine. The method includes feeding an aqueous urea exhaust treatment fluid including biuret to a filter including a filter element, filtering the aqueous urea exhaust treatment fluid using the filter element, providing the filtered aqueous urea exhaust treatment fluid to an injector, and dosing the filtered aqueous urea exhaust treatment fluid into the exhaust, wherein the filter element includes an adsorbent material having a biuret conversion catalyst impregnated therein, and the filtering includes adsorbing the biuret from the aqueous urea exhaust treatment fluid with the adsorbent material, and converting the biuret into a material useful for exhaust after-treatment or into a material that is innocuous.

Lastly, the present disclosure provides a filter for removing biuret from an aqueous urea exhaust treatment fluid that includes a filter element that includes an adsorbent material configured to adsorb the biuret from the aqueous urea exhaust treatment fluid, and the adsorbent material includes a biuret conversion catalyst that is configured to convert the biuret into a material useful for exhaust after-treatment or into a material that is innocuous to an exhaust after-treatment system.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 schematically illustrates an exhaust after-treatment system according to a principle of the present disclosure;

FIG. 2 illustrates an example filter according to a principle of the present disclosure;

FIG. 3 is a graph illustrating that when biuret is present at increased concentrations in an aqueous urea reagent solution relative to the amount of urea present, the effect on solid deposit formation is increased in comparison to aqueous urea reagent solutions that include lower amounts of biuret;

FIG. 4 is a graph illustrating the effect on deposit formation when the amount of biuret in the aqueous urea reagent is decreased using a filter according to the present disclosure; and

FIG. 5 is a graph similar to FIG. 4 illustrating the effect on deposit formation when the amount of biuret in the aqueous urea reagent is decreased using a filter according to the present disclosure, but includes additional data associated with increased reaction times.

Corresponding reference numerals indicate corresponding parts throughout the several views of the drawings.

### DETAILED DESCRIPTION

Example embodiments will now be described more fully with reference to the accompanying drawings.

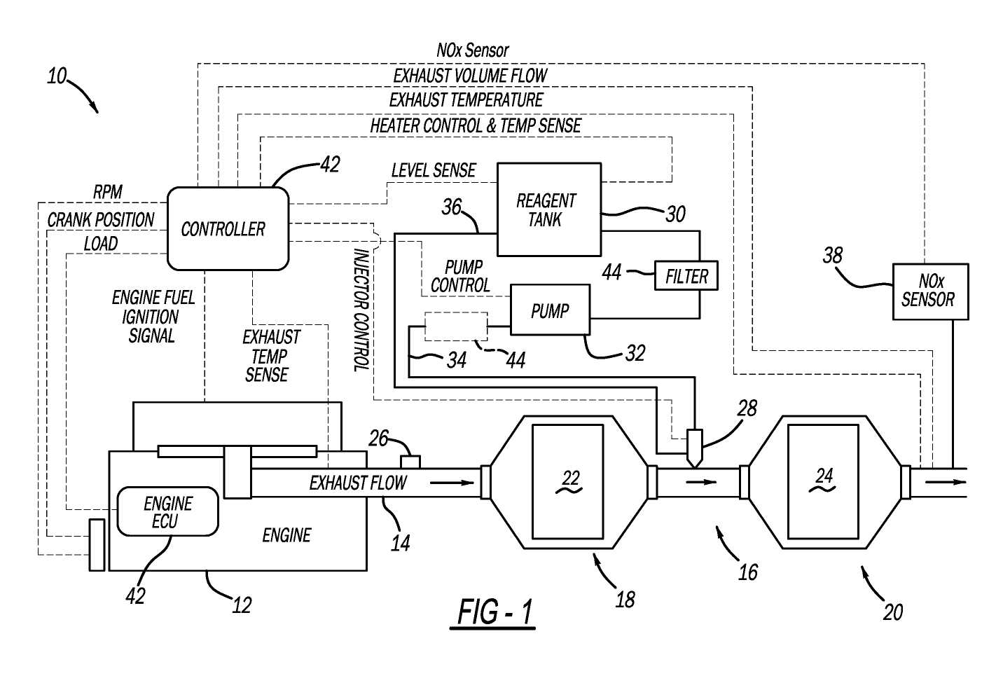

FIG. 1 schematically illustrates an exhaust system 10 for a vehicle according to the present disclosure. Exhaust system 10 can include at least an engine 12 in communication with a fuel source (not shown) that, once consumed, will produce exhaust gases that are discharged into an exhaust passage 14 having an exhaust after-treatment system 16. Downstream from engine 12 can be disposed a pair of exhaust treatment components 18 and 20, which can include catalyst-coated substrates or filters 22 and 24. Catalyst-coated substrate or filter 22 can be either a diesel particulate filter (DPF) or a diesel oxidation catalyst (DOC), while substrate or filter 24 is preferably a selective catalytic reduction (SCR) component. Alternatively, substrate 22 can be an SCR component, and substrate 24 can be a lean NOₓ catalyst, an ammonia slip catalyst, or any other type of exhaust treatment device known to one skilled in the art. If a DPF is used, it may be catalyst-coated (e.g., DOC catalyst-coated). In the illustrated embodiment, filter 22 is a DOC component or DPF component, and filter 24 is an SCR component.

Although not required by the present disclosure, exhaust after-treatment system 16 can further include components such as a thermal enhancement device or burner 26 to increase a temperature of the exhaust gases passing through exhaust passage 14. Increasing the temperature of the exhaust gas is favorable to achieve light-off of the catalyst (if any) in the exhaust treatment components 18 and 20 in cold-weather conditions and upon start-up of engine 12, as well as initiate regeneration of the exhaust treatment component 18 when the exhaust treatment substrate 22 or 24 is a DPF.

To assist in reduction of the emissions produced by engine 12, exhaust after-treatment system 16 can include a reagent dosing system including a metering device or injector 28 for periodically dosing an exhaust treatment fluid into the exhaust stream. As illustrated in FIG. 1, injector 28 can be located upstream of SCR component 24, and is operable to inject an aqueous urea exhaust treatment fluid into the exhaust stream. In this regard, injector 28 is in fluid communication with a reagent tank 30 and a pump 32 by way of inlet line 34 to dose an exhaust treatment fluid such as aqueous urea into the exhaust passage 14 upstream of exhaust treatment components 18 and 20. Injector 28 can also be in communication with reagent tank 30 via return line 36. Return line 36 allows for any exhaust treatment fluid not dosed into the exhaust stream to be returned to reagent tank 30. Flow of the exhaust treatment fluid through inlet line 34, injector 28, and return line 36 also assists in cooling injector 28 so that injector 28 does not overheat. Although not illustrated in the drawings, injector 28 can be configured to include a cooling jacket that passes a coolant around injector 28 to cool it.

The amount of exhaust treatment fluid required to effectively treat the exhaust stream may vary with load, engine speed, exhaust gas temperature, exhaust gas flow, engine fuel injection timing, desired NOₓ reduction, barometric pressure, relative humidity, EGR rate and engine coolant temperature. A NOₓ sensor or meter 38 may be positioned downstream from exhaust treatment components 18 and 20. NOₓ sensor 38 is operable to output a signal indicative of the exhaust NOₓ content to an engine control unit 40. All or some of the engine operating parameters may be supplied from engine control unit 40 via the engine/vehicle databus to a reagent electronic dosing controller 42. The reagent electronic dosing controller 42 could also be included as part of the engine control unit 40. Exhaust gas temperature, exhaust gas flow and exhaust back pressure and other vehicle operating parameters may be measured by respective sensors, as indicated in FIG. 1.

The amount of exhaust treatment fluid required to effectively treat the exhaust stream can also be dependent on the size of the engine 12. In this regard, while the embodiment illustrated in FIG. 1 is generally used for a vehicle such as an automobile, it should be understood that the teachings of the present disclosure are also applicable to large-scale diesel engines used in locomotives, marine applications, and stationary applications that can have exhaust flow rates that exceed the capacity of a single injector 28. Accordingly, although only one injector 28 is illustrated for dosing exhaust treatment fluid, it should be understood that multiple injectors 28 for reagent injection are contemplated by the present disclosure.

Dosing of the aqueous urea exhaust treatment fluid into the exhaust stream passing through exhaust passage 14 may cause solid deposits to form in exhaust passage 14. The formation of these solid deposits is undesirable in that the solid deposits can, potentially, form to an extent that the exhaust passage 14 becomes clogged and create undesirable backpressure in the exhaust system. In addition, the solid deposits may form on substrates 22 and 24, which prevent sufficient contact between the engine exhaust and the catalyzed substrates to effect oxidation or reduction from occurring when the engine exhaust passes through the filters, and potentially prevent the engine exhaust from passing through the filters 22 and 24. One of the materials typically contained in the urea exhaust treatment fluid that acts as a precursor to the formation of the solid deposits is biuret. Commercially available aqueous urea exhaust treatment fluids such as AdBlue® allow for up to 0.3 wt % of biuret (ISO 22241). While the amount of biuret in the commercially available aqueous urea exhaust treatment fluids may be lower than 0.3 wt %, the potential for solid deposit formation remains. The present disclosure, therefore, is directed to further reducing the amount of biuret in the aqueous urea exhaust treatment fluid before being dosed into the exhaust passage 14.

As shown in FIG. 1, exhaust after-treatment system 14 includes a filter 44 located between reagent tank 30 and pump 32. Alternatively, as shown in phantom in FIG. 1, filter 44 can be located between pump 32 and injector 28. In other alternative configurations (not illustrated), filter 44 can be located within reagent tank 30 or located upstream of reagent tank 30. Filter 44 is configured to filter out particulate from the aqueous urea exhaust treatment fluid before passing through injector 28. Further, according to the present disclosure, filter 44 is configured to adsorb biuret from the aqueous urea exhaust treatment fluid.

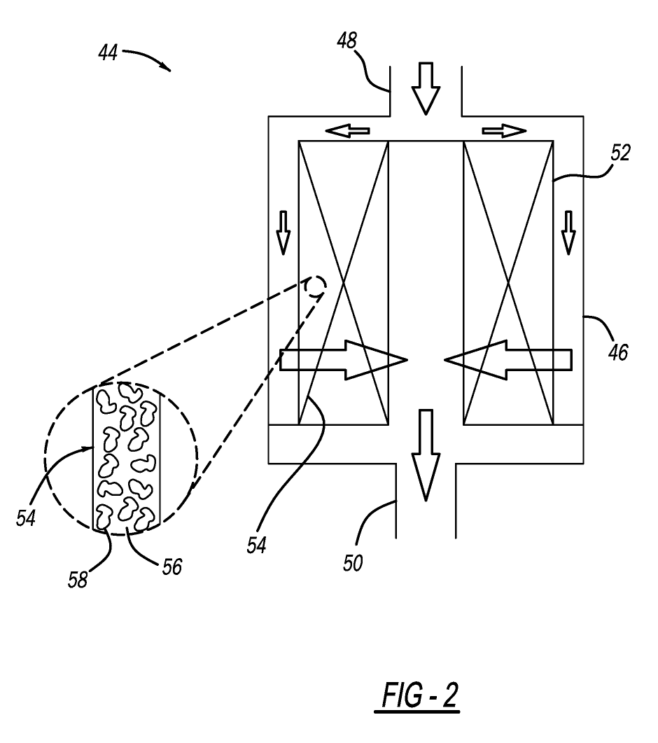

Now referring to FIG. 2, the aqueous urea exhaust treatment fluid filter 44 includes a housing 46 having an inlet 48 that receives the aqueous urea exhaust treatment fluid from reagent tank 30 and an outlet 50 that communicates the filtered aqueous urea exhaust treatment fluid to injector 28. The removal of the solid particulate from the aqueous urea exhaust treatment fluid in which the particulates are mixed is typically accomplished by means of a filter element 52 positioned within housing 46 that is made from a solid material 54 having a plurality of pores of small cross-sectional size extending therethrough, which may be interconnected.

The solid material 54 is permeable to the fluid which flows through the solid material 54, and capable of restraining most or all of the particulates mixed in the fluid. The particulates are collected on the inlet surfaces 56 of the solid material 54 and/or within the pores 58 of the solid material. The minimum cross-sectional size of some or all of the pores can be larger than the size of some or all of the particulates to be removed from the fluid, but only to the extent that significant or desired amounts of sufficiently large particulates become trapped on or within the filter element 52 during the transit of contaminated fluid. As the mass of collected particulates increases, the flow rate of the fluid through the filter element 52 generally decreases to an undesirable level. The filter element 52 is then either discarded as a disposable, replaceable element or regenerated by suitably removing the collected particulates so that it may be reused.

According to the present disclosure, the filter element 52 is formed from a solid material 54 that is configured to chemically adsorb the biuret from the aqueous urea exhaust treatment fluid and then convert the biuret into a material that is advantageous to exhaust after-treatment or innocuous. That is, the solid material 54 includes a biuret adsorbent material and biuret conversion catalyst that converts the biuret into a material into useful or innocuous material. The filter element 52 can be entirely formed from the solid material 54, or the filter element can be formed of a conventional filter material such as polypropylene and then coated with the solid material 54. Regardless which configuration is selected, it should be understood that filter element 52 is designed to both filter out particulates and remove biuret from the aqueous urea exhaust treatment fluid.

The biuret adsorbent material can be selected from natural and synthetic adsorbents, amorphous and crystalline adsorbents, organic and inorganic adsorbents, and acidic, neutral and basic adsorbent materials. The term “adsorbents” is used herein in its conventional sense to denote solid materials that retain one or more components of a solution predominantly, if not exclusively, by mutual physical-chemical attraction. The biuret conversion catalyst can be any alkali or alkaline earth metal oxide, hydroxide, or carbonate.

Example inorganic biuret adsorbent materials include natural and synthetic, amorphous and crystalline oxides, such as silica, oxides of metals such as beryllium, magnesium, calcium, boron, aluminum, gallium, and the like (e.g., alumina, magnesia, beryllia, borax, magnesium silicates, magnesium hydrogen silicates, calcium silicates, aluminosilicates and mixtures or coprecipitates of such oxides). In addition, suitable adsorbents can be obtained by impregnating a porous substrate with one or more of such polar adsorbents, and the polar adsorbent or impregnated adsorbent, as the case may be, can be acid or caustic treated or calcined to modify its physical or chemical properties. When calcination is employed, however, relatively low temperatures are presently preferred since extreme temperatures (e.g. 800 C. and above) can dehydroxylate adsorbents and convert them to relatively non-polar materials. Examples of suitable polar inorganic adsorbents include silica gel, boehmite alumina, Florisil, Magnesol, Silicalite, silica-beryllia cogels, clays such as montmorillonite, halloysite, kaolinite, diatomaceous earth, celite, kiesselguhr, and organo-clays such as derivatives of montmorillonite which have been exchanged with quaternary ammonium ions to form bentones.

Example biuret organic adsorbents include oxidized carbons, natural and synthetic polymers which contain pendant polar groups such as hydroxyl, carboxyl, sulfate, sulfite, amino, amido, thiol, thio, oxy, phosphate, phosphite, etc. including homo-, co-, graft, and substituted (chemically modified) polymers. Specific organic adsorbents include charcoal which has been oxidized at temperature of less than about 400 C, untreated or acid and/or caustic-treated cellulosic matter (e.g., cotton, paper, sawdust, dehydrated plant matter, and other cellulosic material), polyacrylates such as polymers of acrylic acid, ethylhexylacrylate, hydroxyethylacrylate, methacrylic acid, ethyl methacrylate, and the like, phenolics such as phenolformaldehyde polymers, polyethylene thiols, and polycaprolactam. Particularly practical organic adsorbents include cellulose and the acrylate polymers due, primarily, to their availability and relatively low cost.

As noted above, the biuret conversion catalyst includes any alkali or alkaline earth metal oxides (e.g., lithium, sodium, potassium, cerium, and rubidium metals or compounds or any combination thereof), hydroxides (e.g., sodium and potassium hydroxides, hydroxide precursors, and their combinations), and carbonates. Other bases or precursors such as calcium, magnesium, strontium, and barium metals and compounds thereof may also be used.

When these materials are used as the biuret conversion catalyst, the biuret may be converted into urea, or some other type of innocuous substance. To ensure that the biuret is adequately converted to urea or some other type of innocuous substance, the reagent exhaust treatment fluid should be at a temperature sufficient to allow for the catalysis to occur. For example, the reagent exhaust treatment fluid can be at a temperature that ranges between 40 C to 60 C. Regardless, to ensure that the reagent exhaust treatment fluid is at the desired temperature for catalysis, reagent tank 30 may include a heating device (not shown). When the biuret is converted to urea, the additional urea can be used for SCR. The biuret conversion catalyst can be impregnated into the biuret adsorbent material. With this configuration, any accumulated biuret in filter 44 can be converted to urea such that filter 44 is automatically regenerated during use thereof. Regardless, filter 44 is designed for use up to 2000 hours and preferably 5000 hours such that filter 44 can be inspected and/or replaced at the normal required maintenance interval of exhaust after-treatment system 16.

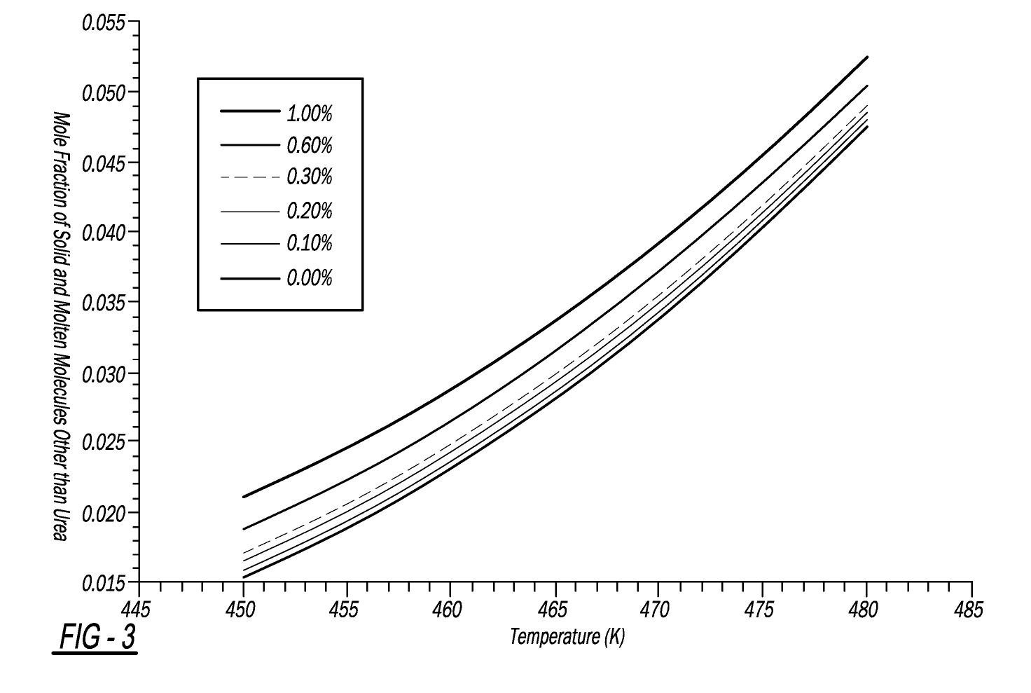

FIGS. 3-5 are graphs illustrating the calculated effect biuret has on solid deposit formation in an exhaust after-treatment system that utilizes an aqueous urea reagent. FIG. 3 illustrates that when biuret is present at increased concentrations in the aqueous urea reagent solution, the calculated effect on solid deposit formation is increased in comparison to aqueous urea reagent solutions that include lower amounts of biuret. It should also be noted that the amount of solid deposits that form as a result of biuret being present in the aqueous urea reagent is calculated to be increased as exhaust temperature increases over the range of temperatures shown.

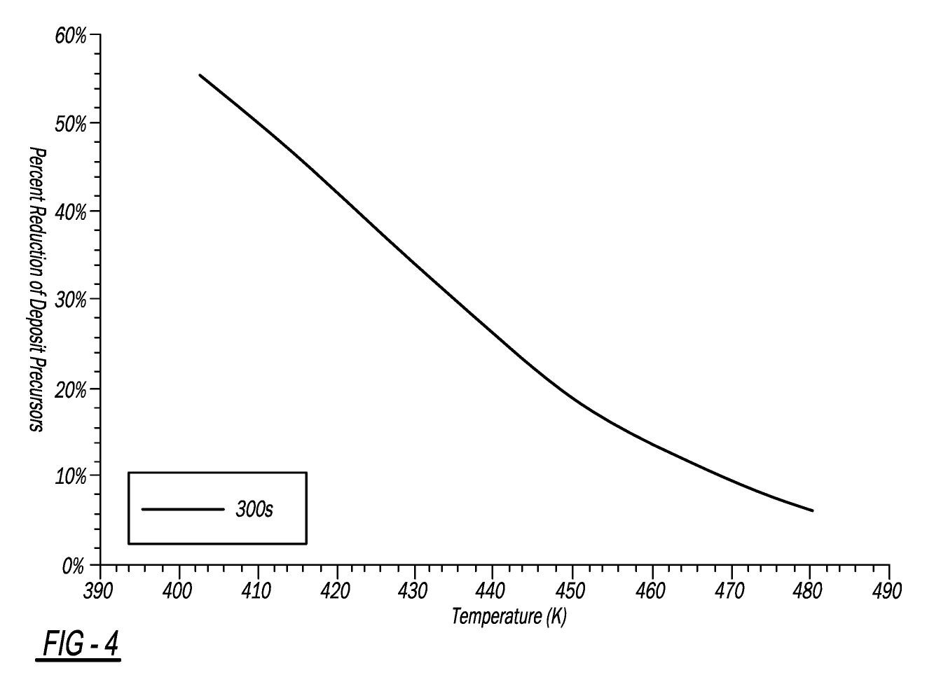

FIG. 4 is a graph illustrating the calculated effect on deposit formation when the amount of biuret in the aqueous urea reagent solution is decreased using a filter according to the present disclosure where the biuret is adsorbed and/or converted into either urea or an innocuous substance. As can be seen in FIG. 4, when the biuret concentration is reduced by filtration from 0.30% to 0.09%, it is calculated that solid deposit formation is drastically reduced at amounts greater than 50% at low temperature. It is evident, therefore, that removal of biuret from the aqueous urea reagent using a filter according to the present disclosure is calculated to be beneficial in substantially minimizing solid deposit formation in the exhaust after-treatment system.

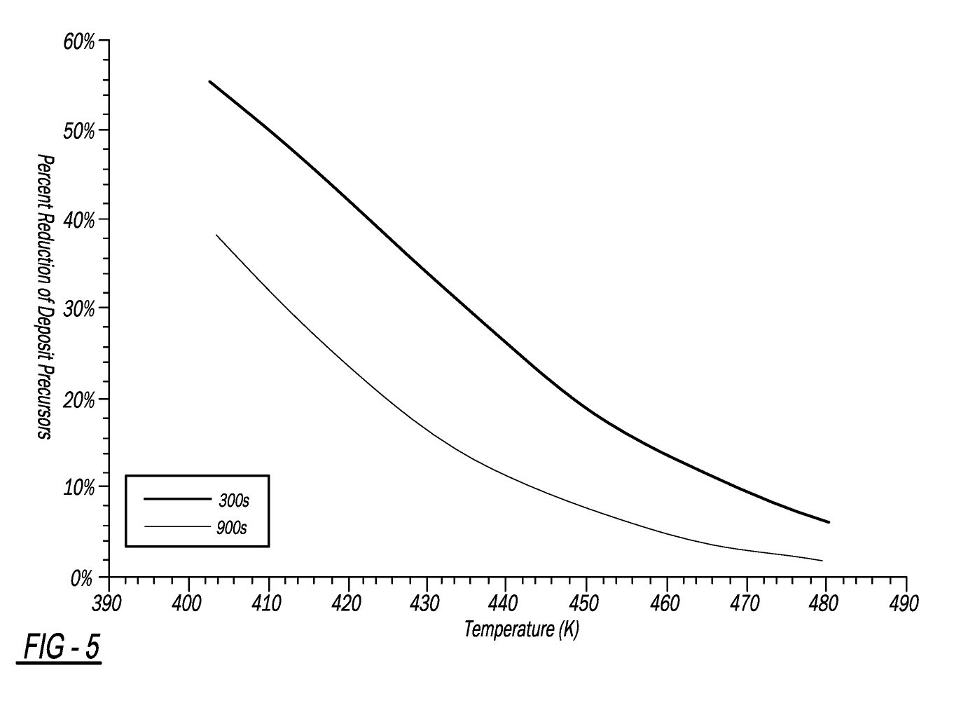

FIG. 5 is similar to FIG. 4, but additionally shows that even at increased reaction times (i.e., 900 s versus 300 s), the reduction of biuret concentration in the aqueous urea reagent using a filter according to the present disclosure is calculated to be effective as substantially minimizing solid deposit formation in the exhaust after-treatment system.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An exhaust after-treatment system for treating an exhaust produced by an engine, comprising:
an exhaust passage in communication with the engine and configured to carry the exhaust;
an injector configured to dose an aqueous urea exhaust treatment fluid into the exhaust passage;
a tank in communication with the injector and configured to provide the aqueous urea exhaust treatment fluid to the injector; and
a filter located between the tank and the injector that is configured to remove impurities from the aqueous urea exhaust treatment fluid,
wherein one of the impurities of the aqueous urea exhaust treatment fluid is biuret, and
the filter includes an adsorbent material configured to adsorb the biuret from the aqueous urea exhaust treatment fluid, and the filter includes a biuret conversion catalyst impregnated in the adsorbent material that is configured to convert the biuret into a material useful for exhaust after-treatment or into a material that is innocuous to the exhaust after-treatment system.

2. The exhaust after-treatment system according to claim 1, wherein the filter includes a filter element, and the filter element is formed of the adsorbent material that includes the biuret conversion catalyst.

3. The exhaust after-treatment system according to claim 1, wherein the filter includes a filter element, and the filter element is formed of a filter material that is coated with the adsorbent material that includes the biuret conversion catalyst.

4. The exhaust after-treatment system according to claim 1, wherein the adsorbent material is at least one material selected from the group consisting of natural and synthetic adsorbent materials, amorphous and crystalline adsorbent materials, organic and inorganic adsorbent materials, and acidic, neutral and basic adsorbent materials.

5. The exhaust after-treatment system according to claim 4, wherein the biuret conversion catalyst is a material selected from the group consisting of alkali or alkaline earth metal oxides, hydroxides, and carbonates.

6. The exhaust after-treatment system according to claim 5, wherein alkali or alkaline earth metal oxides, hydroxides, and carbonates include at least one material selected from the group consisting lithium, sodium, potassium, cerium, and rubidium metals or compounds or any combination thereof.

7. The exhaust after-treatment system according to claim 1, further comprising an SCR exhaust after-treatment component in the exhaust passage downstream from the injector.

8. A method for treating an exhaust produced by an engine, comprising:
feeding an aqueous urea exhaust treatment fluid including biuret to a filter including a filter element;
filtering the aqueous urea exhaust treatment fluid using the filter element;
providing the filtered aqueous urea exhaust treatment fluid to an injector; and
dosing the filtered aqueous urea exhaust treatment fluid into the exhaust
wherein the filter element includes an adsorbent material having a biuret conversion catalyst, and
the filtering includes adsorbing the biuret from the aqueous urea exhaust treatment fluid with the adsorbent material, and converting the biuret into a material useful for exhaust after-treatment or into a material that is innocuous.

9. The method according to claim 8, wherein the filter element is formed of a filter material that is coated with the adsorbent material having the biuret conversion catalyst.

10. The method according to claim 8, wherein the adsorbent material is at least one material selected from the group consisting of natural and synthetic adsorbent materials, amorphous and crystalline adsorbent materials, organic and inorganic adsorbent materials, and acidic, neutral and basic adsorbent materials.

11. The method according to claim 8, wherein the biuret conversion catalyst is a material selected from the group consisting of alkali or alkaline earth metal oxides, hydroxides, and carbonates.

12. The method according to claim 11, wherein alkali or alkaline earth metal oxides, hydroxides, and carbonates include at least one material selected from the group consisting lithium, sodium, potassium, cerium, and rubidium metals or compounds or any combination thereof.

13. The method according to claim 8, wherein the filtering occurs at a temperature in the range of 40 C to 60 C.

14. A filter for removing biuret from an aqueous urea exhaust treatment fluid comprising a filter element that includes an adsorbent material configured to adsorb the biuret from the aqueous urea exhaust treatment fluid, and includes a biuret conversion catalyst in the adsorbent material that is configured to convert the biuret into a material useful for exhaust after-treatment or into a material that is innocuous to an exhaust after-treatment system.

15. The filter according to claim 14, wherein the filter element is formed of the adsorbent material having the biuret conversion catalyst.

16. The filer according to claim 14, wherein the filter element is formed of a filter material that is coated with the adsorbent material having the biuret conversion catalyst.

17. The filter according to claim 14, wherein the adsorbent material is at least one material selected from the group consisting of natural and synthetic adsorbent materials, amorphous and crystalline adsorbent materials, organic and inorganic adsorbent materials, and acidic, neutral and basic adsorbent materials.

18. The filter according to claim 14, wherein the biuret conversion catalyst is a material selected from the group consisting of alkali or alkaline earth metal oxides, hydroxides, and carbonates.

19. The filter according to claim 18, wherein alkali or alkaline earth metal oxides, hydroxides, and carbonates include at least one material selected from the group consisting lithium, sodium, potassium, cerium, and rubidium metals or compounds or any combination thereof.
